(19) United States
(12) Patent Application Publication
(10) Pub. No.: US 2019/0021642 A1
(43) Pub. Date: Jan. 24, 2019

(54) ULTRASONIC VESTIBULAR ANALYSIS

(71) Applicant: UNIVERSITY OF UTAH RESEARCH FOUNDATION, Salt Lake City, UT (US)

(72) Inventors: Richard D. Rabbitt, Salt Lake City, UT (US); Marta M. Iversen, Salt Lake City, UT (US)

(21) Appl. No.: 16/070,218

(22) PCT Filed: Jan. 12, 2017
(86) PCT No.: PCT/US17/13251

§ 371 Date: Jul. 13, 2018

Related U.S. Application Data
(60) Provisional application No. 62/278,838, filed on Jan. 14, 2016.
(60) Provisional application No. 62/436,322, filed on Dec. 19, 2016.

(51) Int. Cl. A61B 5/00; A61B 5/16; A61B 5/11

(52) U.S. Cl. CPC A61B 5/4023; A61B 5/0048; A61B 5/6831; A61B 5/1104; A61B 5/6814; A61B 5/163

(57) ABSTRACT

A system for diagnosing vestibular otolith function can comprise an ultrasonic generator that is configured to direct ultrasonic waves towards vestibular organs with a patient's ear. The system can also comprise a response capture device that is configured to capture patient response to the ultrasonic waves.

### CROSS-REFERENCE TO RELATED APPLICATIONS

This application claims the benefit of and priority to U.S. Provisional Patent Application Ser. No. 62/278,838 filed on Jan. 14, 2016 entitled “ULTRASONIC VESTIBULAR ANALYSIS,” and to U.S. Provisional Patent Application Ser. No. 62/436,322 filed on Dec. 19, 2016 entitled “ULTRASONIC VESTIBULAR ANALYSIS.” Each of the above referenced applications are expressly incorporated herein by reference in their entirety.

### GOVERNMENT RIGHTS

This invention was made with government support under R01 DC011481, R01 DC006685 and R01 DC012060 awarded by the National Institutes of Health. The government has certain rights in the invention.

### BACKGROUND OF THE INVENTION

As many as 69 million Americans have experienced some form of vestibular dysfunction. According to the National Institute on Deafness and Other Communication Disorders (NIDCD), a further 4% (8 million) of American adults report a chronic problem with balance, while an additional 1.1% (2.4 million) report a chronic problem with dizziness alone. Eighty percent of people aged 65 years and older have experienced dizziness. BPPV, the most common vestibular disorder, is the cause of approximately 50% of dizziness in older people. Overall, vertigo from a vestibular problem accounts for a third of all dizziness and vertigo symptoms reported to health care professionals.

Various methods for stimulating vestibular organs for the purpose of testing and diagnosing vestibular dysfunction, restoring vestibular sensory inputs, or providing vestibular afferent signals to downstream neural circuits are known in the art. However, many of these methods are expensive, invasive, and for diagnostic tests can require a high level of expertise to perform and properly interpret. Accordingly, there are a number of problems in the art relating to vestibular dysfunction that can be addressed.

### BRIEF SUMMARY

Embodiments disclosed herein comprise systems, methods, and apparatus configured to provide controlled stimuli to vestibular organs to test vestibular function or control vestibular neural signals sent by the ear to the brain. In particular, disclosed embodiments comprise an ultrasonic generator that is configured to focus packets of ultrasound energy to targeted vestibular organs within a patient's inner ear. Disclosed embodiments relating to vestibular diagnostics also comprise a response capture device that is configured to capture patient response to the focused ultrasound stimulus. The captured response can be utilized to provide significant insights into the function of a patient's vestibular organs.

At least one disclosed embodiment comprises a method for analyzing vestibular organ function. The method can comprise stimulating a vestibular organ with ultrasound. Additionally, the method can comprise capturing a patient response to the ultrasound energy.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above recited and other advantages and features of the invention can be obtained, a more particular description of the invention briefly described above will be rendered by reference to specific embodiments thereof, which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an ultrasonic transducer to focus ultrasound energy or packets on target vestibular organs in accordance with implementations of the present invention;

FIG. 2 illustrates vestibular organs receiving focused ultrasound energy in accordance with implementations of the present invention; and

FIG. 3 illustrates a flowchart of steps in a method for analyzing vestibular organs in accordance with implementations of the present invention.

FIG. 4A depicts an embodiment of a low-intensity focused ultrasound (LiFU) waveforms for pulsed ultrasound stimuli. Insets show individual cycles of 5 MHz ultrasound inside the envelope.

FIG. 4B depicts an embodiment of a LiFU waveforms for continuous ultrasound stimuli. Insets show individual cycles of 5 MHz ultrasound inside the envelope.

FIG. 4C depicts an embodiment of vestibular labyrinth and stimulation by LiFU in an experimental animal

FIG. 5A depicts an embodiment of low-frequency (LF) sensitive afferent neuron modulating action potential firing rate (spk-s⁻¹) in response to continuous wave amplitude modulated LiFU stimulation of the otolith organ.

FIG. 5B depicts another embodiment of LF sensitive afferent neuron modulated in response to continuous wave amplitude modulated LiFU.

FIG. 5C depicts another embodiment of LF sensitive afferent neuron modulated in response to continuous wave amplitude modulated LiFU.

FIG. 5D depicts another embodiment of LF sensitive afferent neuron modulated in response to continuous wave amplitude modulated LiFU.

FIG. 6A depicts an embodiment of auditory-like (AL) high-frequency sensitive saccular afferent neuron modulated in response to pulsed LiFU, with intervals between adjacent action potentials phase-locking firing rate (spk-s⁻¹) to the LiFU stimulus repetition rate (80 pulses per second, pps).

FIG. 6B depicts another embodiment of AL sensitive saccular afferent neuron modulated in response to pulsed LiFU at various levels of stimulus intensity (V) at 10 pps.

FIG. 7A depicts an embodiment of two-unit recording of afferent neurons innervating the sacculus.

FIG. 7B depicts embodiment of two-unit saccular recording with unit #1 responding to the onset of the LiFU packet with latency t1, and unit #2 responding to the termination of the packet with latency t2.

FIG. 7C depicts another embodiment of two-unit saccular recording with action potentials locked to the onset and termination of the LiFU packet.

FIG. 8A depicts an embodiment of a saccular afferent neural response to pulsed LiFU at 100 pps.

FIG. 8B depicts another embodiment of the same afferent as 8A responding to mechanical displacement of the otoconial mass.

FIG. 8C depicts another embodiment of afferent neural responses to LiFU and direct mechanical stimuli applied together to generate periods of constructive and destructive interference, exciting and silencing the afferent neuron.

FIG. 8D depicts another embodiment of otolith organ action potential (AP) probability in time relative to LiFU and mechanical stimuli presented separately.

FIG. 9A depicts an embodiment of a saccular afferent neuron responding to continuous wave LiFU amplitude modulated at 5.1 Hz.

FIG. 9B depicts an embodiment of a saccular afferent neuron responding to 5 Hz mechanical displacement of the otoconial mass.

FIG. 9C depicts an embodiment of saccular afferent responses during constructive and destructive interference of LiFU and mechanical stimulation of the otolith.

FIG. 9D depicts an embodiment of saccular afferent AP probability in response to sinusoidal mechanical and LiFU stimulation.

### DETAILED DESCRIPTION OF THE PREFERRED EMBODIMENTS

Disclosed embodiments extend to systems, methods, and apparatus configured to stimulate vestibular organs and/or test vestibular function. For example, disclosed embodiments comprise a focused ultrasound transducer that is configured to direct ultrasonic energy or packets towards targeted vestibular organs within a patient's ear. Disclosed embodiments also comprise a response capture device that is configured to capture patient response to the ultrasound energy. The captured response can be utilized to provide significant insights into the function of a patient's vestibular organs.

Accordingly, disclosed embodiments provide a novel test for vestibular otolith function that has potential to supplant currently used clinical tests in this area. Disclosed embodiments, leverage the characteristic that healthy vestibular otolith organs are sensitive to energy delivered by packets of focused ultrasound. In at least one embodiment, ultrasound stimuli stimulate sensory hair cells to evoke action potentials in the vestibular nerve and, among other things, lead to vestibular-evoked myogenic potentials (VEMPs) in the neck and ocular muscles.

The otolith organs are part of the vestibular system that sense linear gravito-inertial acceleration. The two otolith organs in each ear are the utricle and saccule and are oriented in the horizontal and vertical planes to sense accelerations in these respective planes. They consist of a high density, high acoustic-impedance otolithic mass attached to a membrane embedded with hair cells. The semicircular canals make up the rest of the vestibular system and sense angular acceleration in three-dimensions. The otolith organs send information to the brain through spike trains that encode direction, amplitude, timing, and frequency of gravito-intertial accelerations. These vestibular organs are capable of sensing a wide range of frequencies from gravitational (<10 Hz) to auditory (20 Hz-5 kHz).

Vestibular evoked myogenic potentials (VEMPs) are triggered by afferent neural signals originating in the vestibular organs, and are measured clinically to test otolith function. Either bursts of intense air-conducted sound (ACS) or bone-conducted vibrations (BCV) are applied at auditory frequencies and motor outputs of the cervical sternocleidomastoid muscles (cVEMPs) or extraocular muscles (oVEMPs) are measured. VEMP signals reflect function of the otolith organs and the neural circuitry responsible for the corresponding muscle activation. Data suggest that under normal conditions, cVEMPs arise primarily from activation of saccular afferent neurons through ipsilateral motor inputs, while oVEMPS arise from activation of utricular afferent neurons. Both ACS and BCV activate auditory-like (AL) afferent neurons by vibrating hair bundles which leads to the rapid modulation of mechano-electrical transduction (MET) currents and the triggering of action potentials (APs). These AL vestibular afferents are characterized by their irregular inter-spike-intervals (ISI) and ability to fire APs locked to a precise phase of the stimulus. AL afferents respond to low-frequency sinusoidal acceleration with increased gain for increased frequency, and exhibit the ability to phase lock to auditory frequencies exceeding 2 kHz.

Otolith afferents with regular ISIs, however, encode for linear acceleration and head orientation relative to gravity. Additionally, they have constant gains in mid-band frequency range, and do not respond to vibrational stimuli at auditory frequencies. These low-frequency (LF) fibers do therefore not respond to either ACS or BCV stimuli and are not responsible for VEMP signals. In at least one disclosed embodiment, low-intensity focused ultrasound (LiFU) in the 1-5 MHz frequency range can selectively activate AL or LF otolith afferent neurons based on the LiFU stimulus waveform. As such, LiFU could have advantages over conventional methods used to test otolith function or could potentially serve as a selective stimulus to preferentially activate saccular or utricular afferents and their compensatory neural circuitry.

In at least one embodiment, LiFU modulates neural activity transcranially. Further, in at least one embodiment, 1-5 MHz LiFU deposits both thermal and mechanical energy to tissue thereby altering the Gibbs free energy of excitable ion channels, modulating membrane capacitance, and altering intracellular signaling. These mechanisms can lead to either excitation or inhibition of neurons and vestibular hair cells. Disclosed embodiments provide the excitation of vestibular afferents in the otolith organs due to 5 MHz LiFU with the main mechanism being simple momentum transfer from the ultrasound wave to the otolithic mass.

Disclosed embodiments provide significant benefits over conventional vestibular diagnostic and testing methods. For example, disclosed embodiments are capable of stimulating individual vestibular organs with directional precision, thus providing a new level of specificity not previously available. Additionally, unlike traditional VEMP testing, at least one disclosed embodiment avoids exposing the cochlea to loud sounds, which can be uncomfortable and possibly damaging to hearing. Also, at least one disclosed embodiment delivers repeated ultrasound stimuli for evoked response averaging to improve VEMP measurements and vestibular brainstem electrical responses (VBR).

In at least one disclosed embodiment, an ultrasound generator focuses energy towards the vestibular organs and a response capture device captures a patient's response. For example, the ultrasound transducer directs ultrasound stimulus to the outside of the skin in pulses focused to excite specific vestibular organs. The response capture device records ultrasound driven VEMPs, VBRs, compensatory eye movements resulting from the vestibulo-ocular reflex (VOR), or vestibulo-sympathetic reflexes. The response capture device may comprise simple electrodes on the surface of the skin, or invasive electrodes. Alternatively, the response capture device may comprise an optical eye movement recording or sympathetic responses including blood pressure recording or heart rate. Responses can be averaged over many rapid presentations of the ultrasound stimulus to generate a clean stimulus-response waveform. The magnitude and latency of the response can then be used to diagnose function of the organ or, alternatively, to control the response through feedback.

Disclosed embodiments include testing equipment that is inexpensive and easy to acquire. For example, the equipment may comprise a focused ultrasound stimulus transducer, a head strap to aim the probe and hold it against the skin over the bone, a generator to drive the stimulus probe, inexpensive voltage preamplifiers to sample electrical potentials from electrodes on the surface of the skin, and an inexpensive computer or processor to sample, average, and display the data. Cost can be further reduced by incorporating the test into current systems used to perform other vestibular and/or audiometric tests.

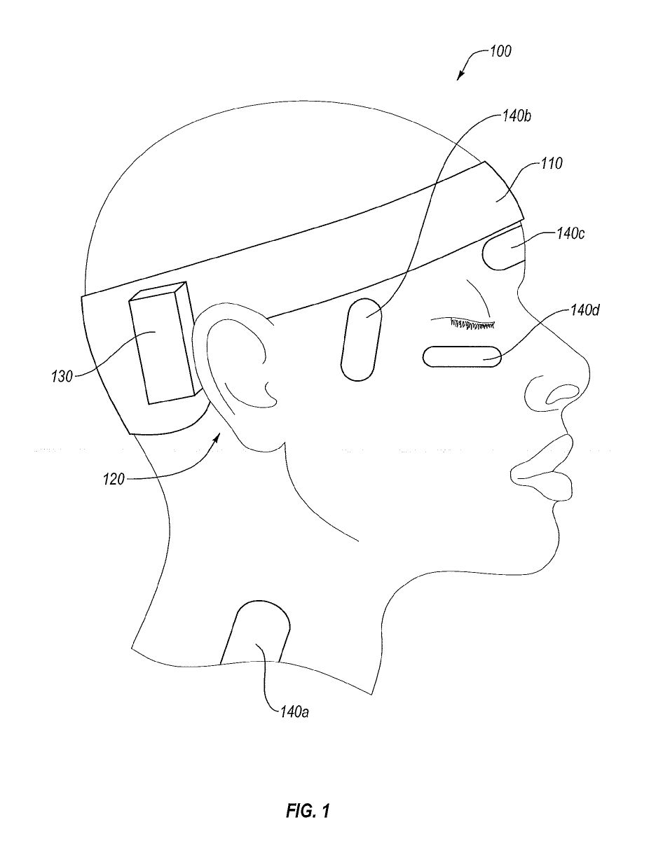

Turning now to the Figures, FIG. 1 illustrates an ultrasonic generator 130 directing ultrasound energy towards vestibular organs in accordance with implementations of the present invention. As depicted, a head strap 110 can be attached to a patient's head 100, such that the ultrasonic generator 130 is positioned near the patient's ear 120. In alternate implementations, the ultrasonic generator 130 is positioned in different locations relative to the ear 120 delivering energy from different directions, and may comprise a different holding device than the depicted head strap 110.

FIG. 1 also depicts various embodiments of response capture devices 140(a-d). In particular, the response capture devices 140(a-d) comprise respective electrodes for measuring VEMPs and or VBRs. While FIG. 1 only depicts electrodes as response capture devices 140(a-d), in various embodiments, alternate or additional response capture devices 140 and techniques can be used. For example, brainstem responses to the ultrasonic waves can be measured, the patient's eye movement can be measured, or any number of other novel and conventional methods can be used to track the patient's response to the ultrasonic stimuli. Based upon the patient's response to the ultrasonic stimuli, various diagnosis and testing can be performed using conventional knowledge. For example, an individual will demonstrate specific VEMPs that are conventionally associated with known patterns and responses.

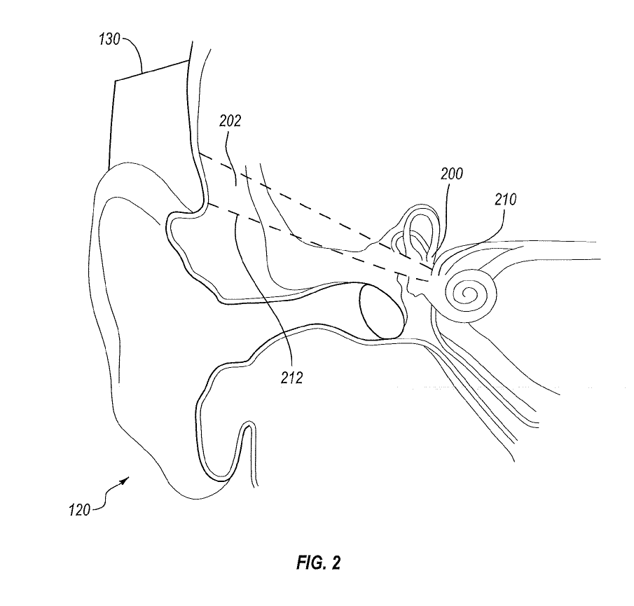

FIG. 2 illustrates vestibular organs receiving ultrasonic stimulation in accordance with implementations of the present invention. As depicted in FIG. 2, the ultrasonic generator 130 focuses ultrasonic waves 202, 212 into the inner ear towards targeted vestibular organs. In at least one implementation, the ultrasonic waves are focused such that the wave 212 is directed towards the saccule 210 to the substantial exclusion of the utricle 200 or the wave 202 is directed to the utricle 200 to the substantial exclusion of the saccule 210. As such, a patient's saccule 210 function can be diagnosed in isolation from their utricle 200 function, and vice versa.

Additionally, in at least one embodiment, the ultrasonic generator 130 directs waves through a patient's cranium bone and tissue, and into the patient's vestibular organs. As such, the ultrasonic generator 130 can be positioned on the outside of the patient's head 100.

Additionally, in at least one embodiment, the ultrasonic generator 130 is configurable to direct sonic waves into the vestibular organs from specific angles or from a specific set of angles. For example, the ultrasonic generator 130 may be configured to stimulate the saccule 210 at a series of different angles such that a clinician can attempt to observe differences in the captured response as they relate to the different directions of stimulation. The changes in angles may stimulate different neurons. In particular, the ultrasonic stimulation causes sensory hair cells oriented in the direction of the traveling ultrasound wave to be preferentially stimulated. The otoconia moves in the direction of the ultrasound wave to stimulate hair cells. In at least one embodiment, changing the angle of ultrasonic stimulation adjusts which hair cells are stimulated.

In at least one embodiment, an ultrasonic generator 130 as described above is incorporated into one or more treatment programs. For example, in at least one implementation, an ultrasonic generator 130 is used to activate vestibular afferent neurons and send useful signals the brain. In some cases, orthostatic intolerance can be treated through ultrasonic activation of vestibular organs.

It has been observed that stimulation of the vestibular organs can control blood pressure and compensate for sudden changes in body orientation relative to gravity. In at least one embodiment, a feedback system is used to control the ultrasonic generator 130. For example, a blood pressure monitor detects when a patient's blood pressure suddenly drops or drops below a threshold. In response to the detection, the ultrasonic generator stimulates the vestibular organs to compensate for the drop in blood pressure. As such, disclosed embodiments use a feedback loop to adjust the ultrasound stimulus on the basis of the captured response, for the purpose of controlling the captured response.

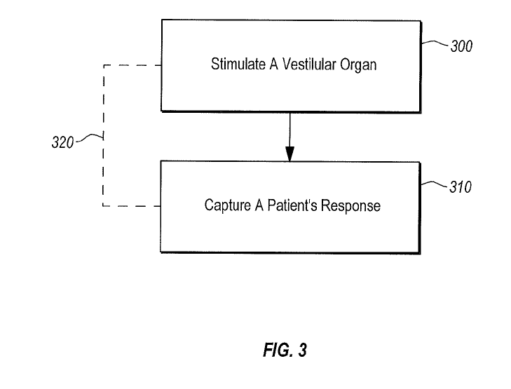

FIG. 3 illustrates a flowchart of steps in an embodiment of a method for analyzing vestibular organs. FIG. 3 shows that a method for testing vestibular organ function includes an act 300 of stimulating an organ with ultrasonic waves. Act 300 can comprise stimulating a vestibular organ with an ultrasonic wave. For example, in FIGS. 1 and 2, and the accompanying description, an ultrasonic generator 130 directs ultrasonic waves to a patient's vestibular organs 200, 210. In at least one implementation, the ultrasonic generator 130 focuses its waves onto a single vestibular organ.

FIG. 3 also shows that the method includes an act 310 of capturing a response. Act 310 can include capturing a patient response to the ultrasonic waves. For example, in FIG. 1 and the accompanying description, VEMP is measured through the use of electrodes 140 attached to the patient. Other systems for capturing a patient's response can also be utilized, such as but not limited to: tracking eye motion, tracking brain stem activity, tracking blood pressure, etc. In some implementations, a feedback control loop 320 is used to adjust the ultrasound stimulus to achieve a desired captured response.

Accordingly, implementations of the present invention provide a novel method and system for testing a patient's vestibular organ function and vestibular neural control of compensatory responses. Such tests can be performed through the cranium bone, without directing loud noises into a patient's ear. Additionally, ultrasonic waves can be focused such that they only interact with a single vestibular organ and do not substantially stimulate others.

### EXAMPLE

The following disclosure relates to an example of an embodiment of ultrasonic vestibular analysis. In particular, at least a portion of the example relates the experimental results related to the use of ultrasonic vestibular analysis on a particular species of fish. One of skill in the art will appreciate that these experimental results are adaptable for use on a human. The examples provided herein are provided merely for the sake of explanation and clarity and do not limit the scope of the invention to a particular embodiment.

As an example of an experimental embodiment, fish were immersed in bubbled seawater containing MS222 (3-aminobenzoic acid ethyl ester, Sigma, 25 g/L) and partially immobilized by an injection of pancuronium bromide (0.05 mg/kg) in the tail muscle. The fish was secured in a plastic tank with their dorsal surface covered by moist tissues. A small (2 cm) dorsal craniotomy was performed to give direct acoustic access to the utricle, saccule, semicircular canals and electrical access to nerve respective branches for recording of action potentials. A polystyrene culture dish with a hole in the bottom was sealed onto the head and filled with optically beneficial fluorocarbon (FC-880, 3M), which allowed for immersion and acoustic coupling of the LiFU transducer face. A 5 MHz spherically focused ultrasound transducer (Olympus, C309-SU P) was driven by a power amplifier (EIN, 240 L RF) and amplitude modulated (Textronix, AFG320) to deliver short pulses of constant amplitude LiFU (pLiFU), or continuous-wave sinusoidally modulated LiFU (cwLiFU). Pulses were delivered at 1-2000 pps (pulse width 20-1000 μs) and continuous waves were applied at 0.1-100 Hz (delivering ˜0-0.4 g equivalent gravito-inertial acceleration to the otolithic mass).

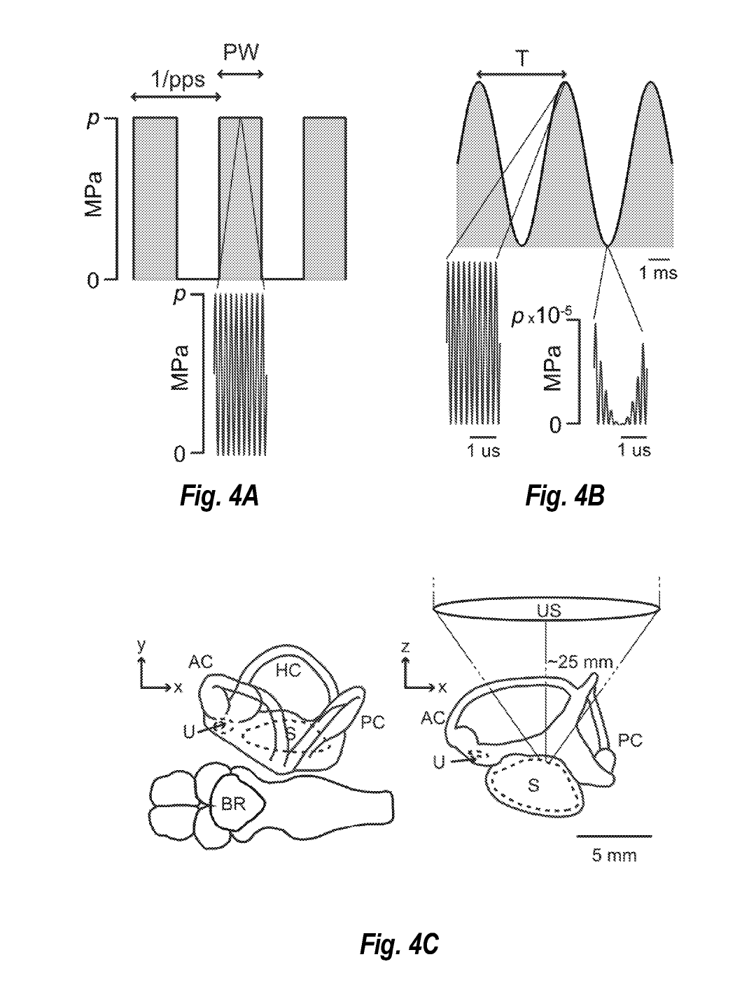

FIG. 4A shows representative waveforms used for pulsed LiFU, and FIG. 4B shows representative waveforms used for continuous LiFU. The transducer was mounted on a micromanipulator at a distance of 1 inch allowing the transducer to be directly focused on individual vestibular organs. FIG. 4C shows the vestibular labyrinth of the fish and the US was applied in the −z-direction (dorsal to ventral).

Mechanical indentation was applied to the semicircular canal ducts or to the otoliths with a pulled and heat-finished glass pipette attached to a piezoelectric actuator and servo controller. Responses to mechanical stimulation were used to simulate physiological motion of the head and characterize afferent neural responses for comparison to responses to LiFU. Single-unit extracellular or intracellular afferent recordings were made with conventional glass electrodes in the nerve branch of the organ of interest.

Vestibular organs were also excised, fixed in 2% paraformaldehyde in 1M PBS, suspended by a force calibrated elastic wire, and exposed to cwLiFU. Acoustic radiation force acting the otolithic mass by LiFU was measured by deflection of the calibrated wire. Temperature was measured using a micro-thermistor to determine the maximum temperature increase due to LiFU application.

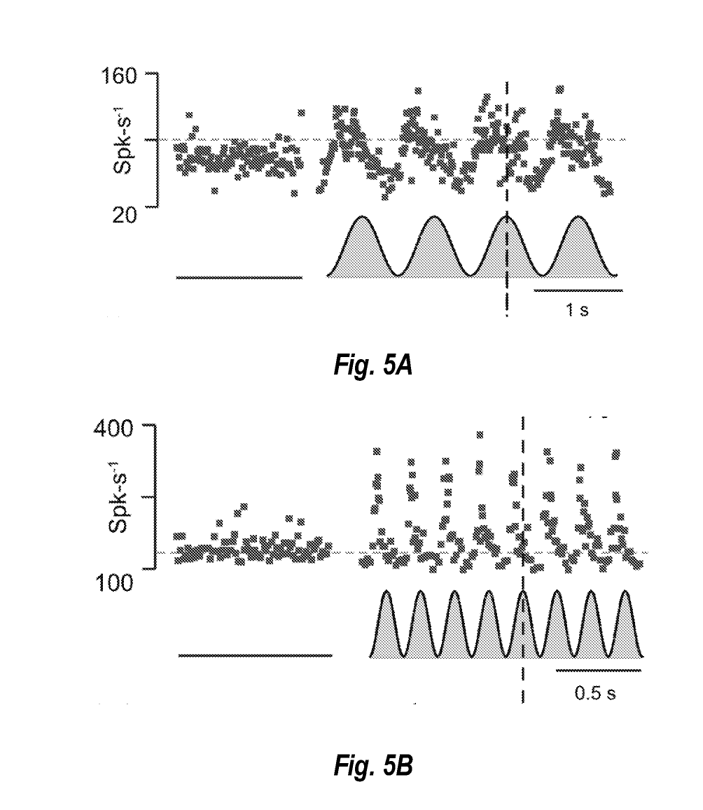

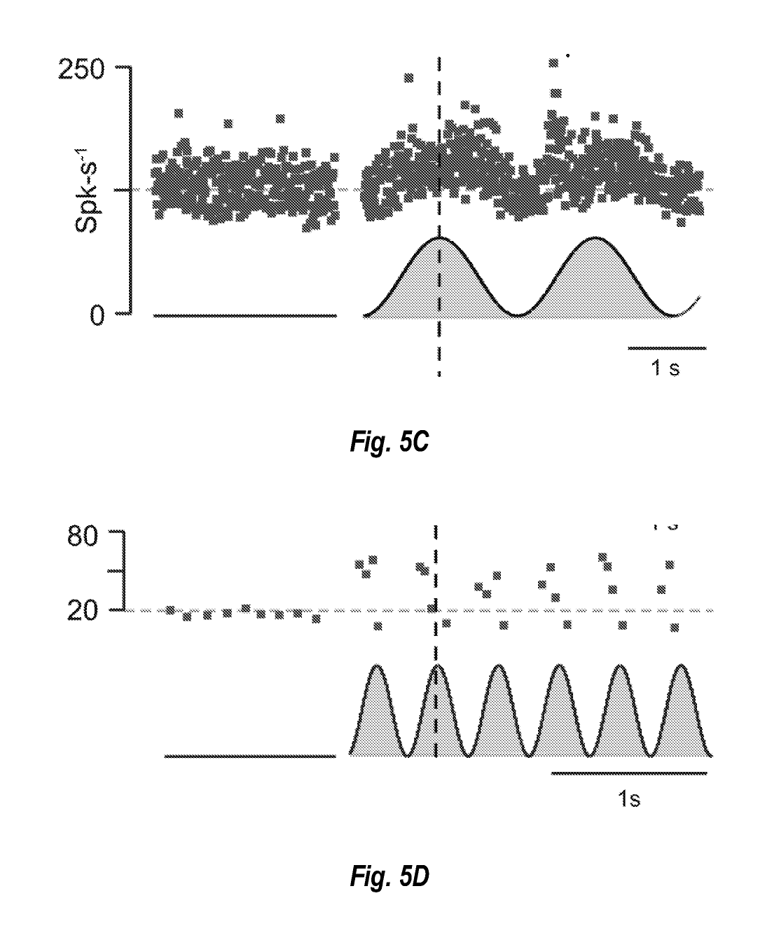

Continuous wave LiFU (cwLiFU) was shown to modulate LF sensitive afferents by generating a sustained force on the otolithic mass. FIGS. 5(A-D) show representative afferents responding to cwLiFU. The afferent neurons in FIGS. 5A and 5B responded with action potential peak firing rate 90° phase advanced, and the afferent neurons in FIGS. 5C and 5D respond with firing rate in phase with the LiFU stimulus. LF afferent neurons responded to cwLiFU with frequency-modulated discharge rates, mimicking responses of these same neurons to changes in orientation relative to gravity or linear acceleration. Unlike sinusoidal linear acceleration, LiFU only generates force in the positive direction of the acoustic wave, thus afferents modulated AP rate in only one direction (e.g. 3G, + above the resting rate). These LF afferents that responded to cwLiFU by changing their discharge rates did not respond to pLiFU.

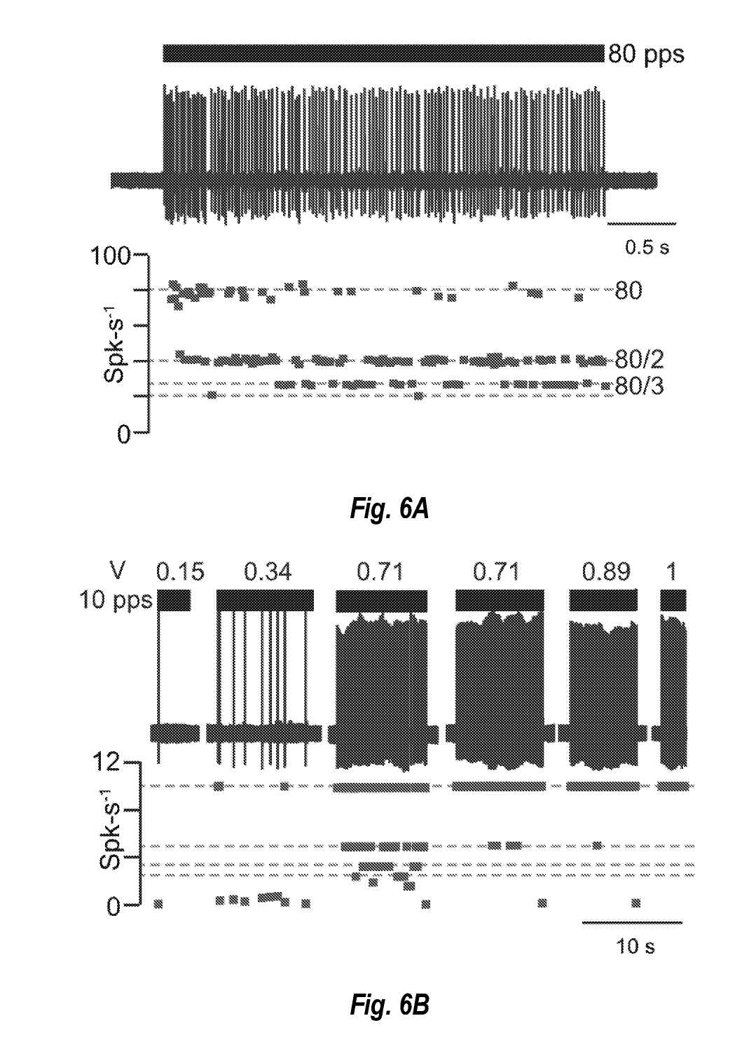

Pulsed LiFU however, was shown to activate AL otolith afferent neurons. The most sensitive units fired an action potential for every LiFU pulse, locking discharge rate to LiFU stimulus rate (pulses per second, pps). Less sensitive units responded with APs at various winding ratios. The unit shown in FIG. 6A responded to 80 pps LiFU initially at a winding ratio of one, then adapted to firing at winding ratios of two and three. The winding ratio approached one as the strength of the stimulus increased as shown in FIG. 6B with pLiFU applied to the saccule at 10 pps. In all afferents, APs were evoked for each LiFU packet and not by individual ultrasound cycles (2000-2500 cycles/packet) within the packet. These AL afferent neurons did not respond to cwLiFU as expected by the nature of AL units.

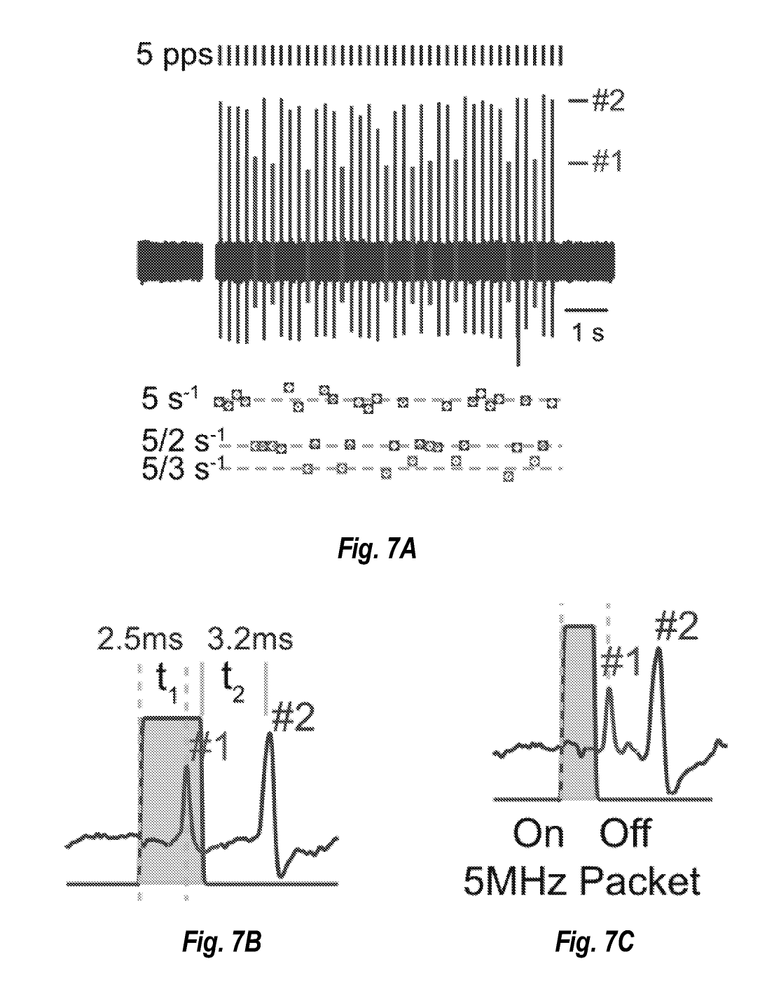

Otolith afferent neurons responded to the rate-of-change of force as shown by the dual-unit recording in FIG. 7A. Some afferent neurons responded to the onset of the ultrasound pulse while others responded to the termination of the ultrasound pulse. For example, Unit #1 in FIGS. 7B and 7C responded to the onset of the LiFU packet with a latency of 2.5 ms and unit #2 responded to the termination of the LiFU packet with a latency of 3.2 ms. FIGS. 7B and 7C utilize different pulse widths to demonstrate the relationship between the onset and termination of the pulse with respect to the neural response. This difference suggests that unit #1 innervated hair cells with dorsal polarity opposite the ventral LiFU beam and unit #2 innervated hair cells with ventral polarity in the same direction of the LiFU beam.

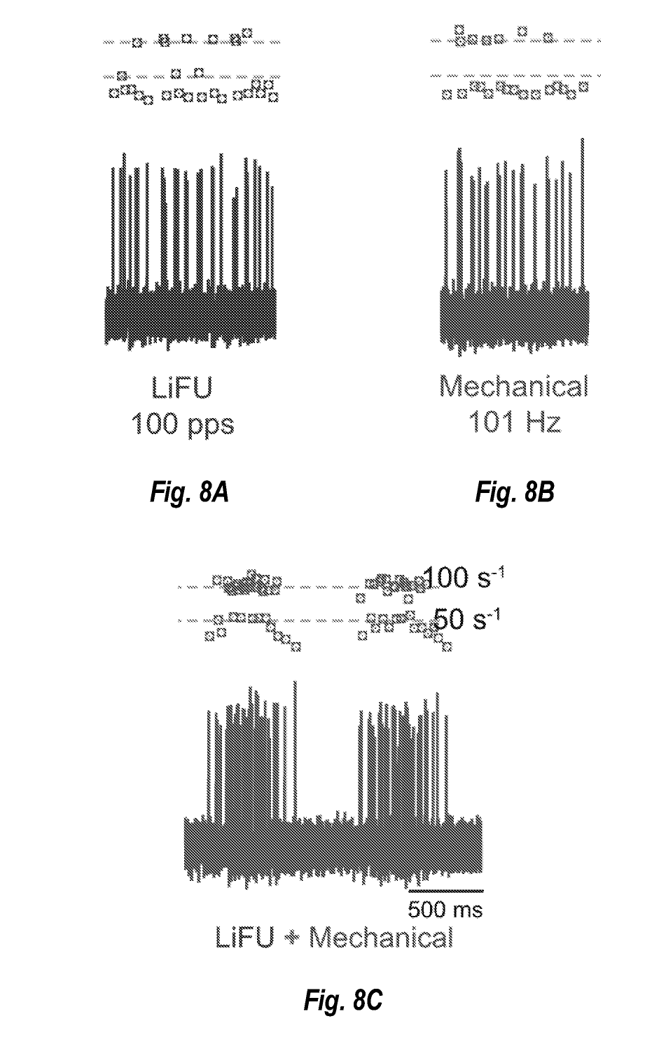

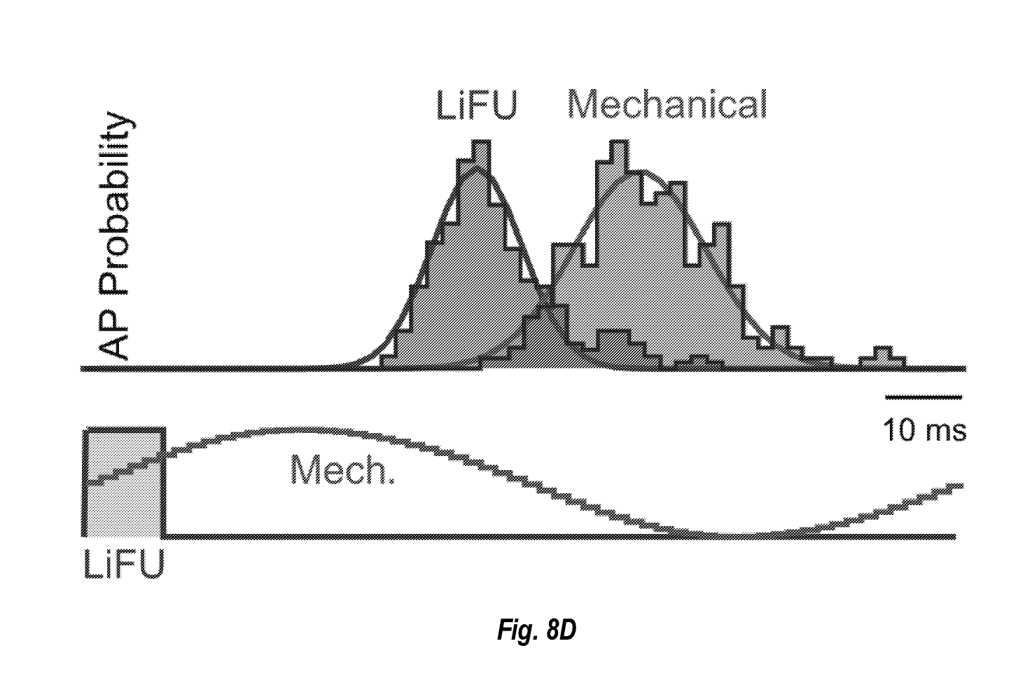

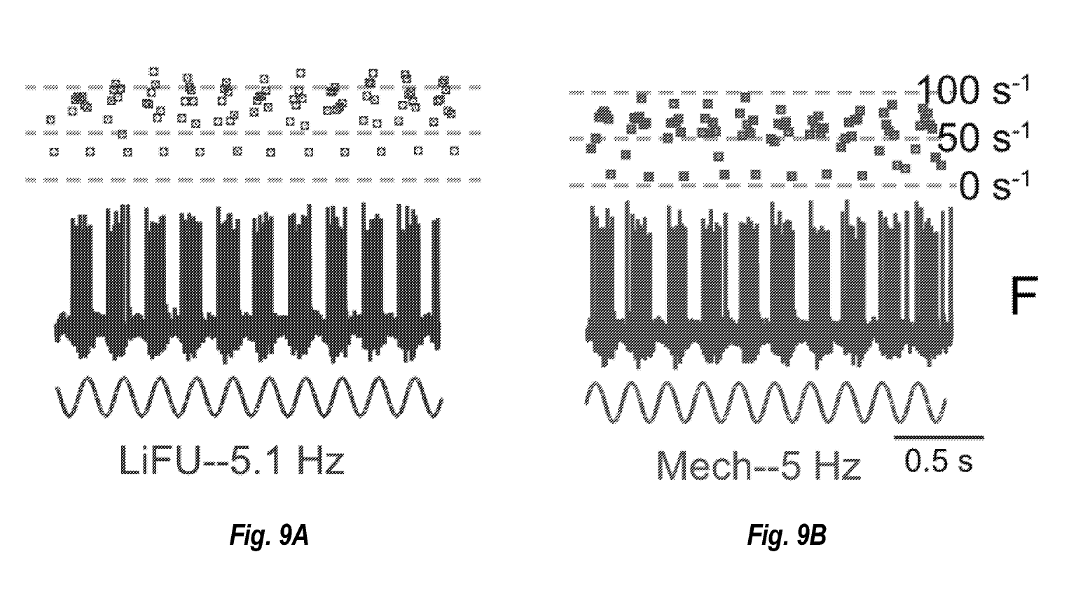

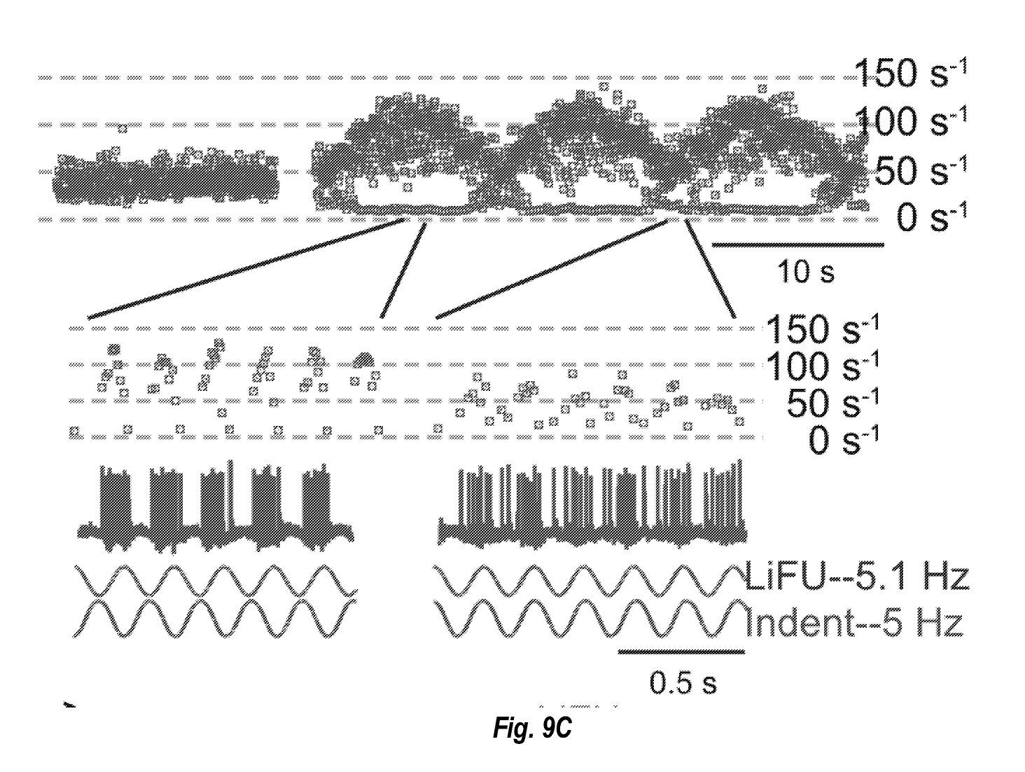

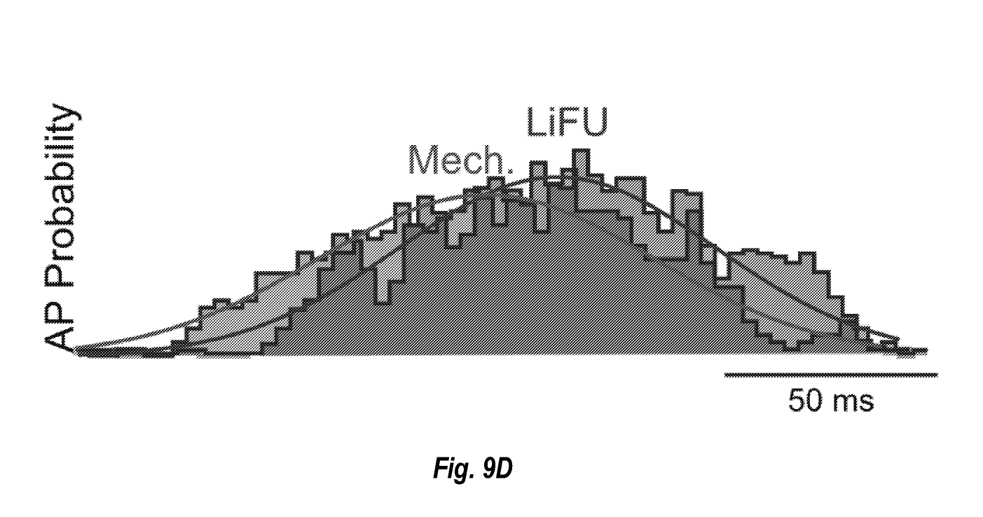

LiFU evoked responses were shown to be equivalent to direct mechanical stimulation with both pulsed and continuous wave configurations. FIGS. 8(A-D) and 9(A-D) show representative neural responses to simultaneous mechanical and LiFU stimulation with pulsed (shown in FIGS. 8(A-D)) and continuous (shown in FIGS. 9(A-D)) LiFU. Both pLiFU at 100 pps and mechanical stimulation at 101 Hz were applied individually (shown in FIGS. 8A and 8B) and simultaneously (shown in FIG. 8C). Simultaneous stimulation showed afferent response at the beating frequency of 1 Hz where the constructive interference of the stimuli occurred. Phase histograms in FIG. 8D show responses with stronger vector strength for LiFU stimulation as expected by difference in the stimulus waveforms.

FIG. 9A shows responses of a saccular afferent neuron to stimulation with cwLiFU at 5.1 Hz and FIG. 9B shows mechanical stimulation at 5 Hz. In FIG. 6C the stimuli are presented together and again beating constructive and destructive interference causes the afferent discharge rate to modulate at the different frequency 0.1 Hz. The phase histograms in FIG. 9D show that the discharge probabilities are similar when continuous wave sinusoidal modulation are used.

In various disclosed embodiments, continuous LiFU is able to mimic responses of afferents to changes in orientation relative to gravity in LF afferent neurons. These otolith neurons are not sensitive to auditory vibrational frequencies and are therefore do not respond to stimuli used in conventional clinical VEMPs testing. Additionally, AL afferent neurons responded to pulsed LiFU, consistent with ACS or BCV stimuli used in VEMP testing. Both responses to LiFU are physiologically relevant and make ultrasound a compelling stimulus for clinical testing and vestibular research.

The LiFU evoked afferent responses were not due to changes in temperature. The temperature change with all stimulus parameters used was less than 1° C. The latency to action potential for pLiFU was also less than 1 ms, much faster than if heat were the mechanism of action. The primary mechanism causing vestibular hair cell response to LiFU is mechanical force generated by the ultrasound acting on the otolithic mass, analogous to physiological gravito-inertial force acting on the mass.

The force measured due to 5 MHz LiFU was shown to be mostly due to reflection of the ultrasound on the otolithic mass. The force on only the semicircular canals was much smaller, and primarily due to absorption of the ultrasound wave rather than reflection off the otolithic mass. The equivalent “G” force would be expected to increase for smaller organs (e.g. mouse, human) because the radiation force scales approximately as frontal area while the mass scales as volume. The human otolith organs are closer in size to the utricle in Opsanus tau. In at least one embodiment, afferent neurons in the semicircular canals modestly responded to LiFU stimulation of the sensory epithelium. Some semicircular canal units do not respond to LiFU and some more sensitive units exhibit low gain responses. The low forces in the excised canals explain why there was much lower sensitivity in the semicircular canals. However, disclosed embodiments are able to elicit some responses so the absorption component of the radiation force can be sufficient for modest activation of the semicircular canals. Additionally, the force in the semicircular canals alone is small enough that it does not damage the sensory apparatus. The mammalian organ of Corti consists of soft tissues with acoustic impedance similar to the canals indicating similar forces would be present when LiFU is focused on the cochlea. Forces generated by LiFU on the otolith organs is an order of magnitude higher because of the large acoustic impedance mismatch at the surface of the calcium carbonate rich otoconial mass.

Accordingly, focused ultrasound provides a new means for selective otolith activation with possible applications in basic science, clinical assessment, and therapeutics.

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

Embodiments of the present invention may comprise or utilize a special-purpose or general-purpose computer system that includes computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A system for use in diagnosing vestibular otolith function comprising:
an ultrasonic generator that is configured to direct ultrasonic energy towards vestibular organs within a patient's ear; and
a response capture device that is configured to capture patient response to the ultrasonic energy.

2. The system as recited in claim 1, wherein the response capture device measures vestibular-evoked myogenic potentials.

3. The system as recited in claim 1, wherein the response capture device measures vestibular-evoked brainstem response.

4. The system as recited in claim 1, wherein the response capture device measures eye movements.

5. The system as recited in claim 1, wherein the response capture device measures body movements or sway.

6. The system as recited in claim 1, wherein the ultrasonic generator is configured to direct ultrasonic energy towards the vestibular saccule to the substantial exclusion of the utricle.

7. The system as recited in claim 1, wherein the ultrasonic generator is configured to direct an ultrasonic energy towards the vestibular utricle to the substantial exclusion of the saccule.

8. The system as recited in claim 1, further comprising a biometric tracking device configured to track a biometric attribute of a patient, wherein the ultrasonic generator is configured to activate in response to a detected biometric attribute.

9. The system as recited in claim 1, wherein the ultrasonic generator is configured to generate ultrasonic energy at 1-10 MHz.

10. The system as recited in claim 9, wherein the ultrasonic generator is configured to generate ultrasonic energy at 5 MHz.

11. The system as recited in claim 1, wherein the signal from the capture device is used in a feedback loop to control the physiological response by adjusting the ultrasound stimulus.

12. The system recited in claim 11, wherein the feedback loop is used to treat orthostatic intolerance.

13. The system recited in claim 11, wherein the feedback loop is used to control eye movements.

14. The system recited in claim 11, wherein the feedback loop is used to control body posture, balance, or sway.

15. The system recited in claim 11, wherein the feedback loop is used to restore, amplify, or otherwise evoke vestibular sensation.

16. The system recited in claim 11, wherein the feedback loop is used treat attacks of vertigo.

17. A method for analyzing vestibular otolith function comprising:
stimulating a vestibular organ with an ultrasonic energy; and
capturing a patient response to the ultrasonic energy.

18. The method as recited in claim 17, further comprising stimulating the vestibular saccule to the substantial exclusion of the utricle.

19. The method as recited in claim 17, further comprising stimulating the vestibular utricle to the substantial exclusion of the saccule.

20. The method recited in claim 17, further comprising a feedback control loop where the ultrasonic stimulus is adjusted on the basis of the captured response for the purpose of controlling the response.
